(19) United States
(12) Patent Application Publication
(10) Pub. No.: US 2024/0302361 A1
(43) Pub. Date: Sep. 12, 2024

(54) MAGNETICALLY ACTIVATED INDIVIDUAL CELLS SORTING

(71) Applicant: THE REGENTS OF THE UNIVERSITY OF CALIFORNIA, Oakland, CA (US)

(72) Inventors: Abdon SEPULVEDA, Los Angeles, CA (US); Victor ESTRADA, Los Angeles, CA (US); Ruoda ZHENG, Los Angeles, CA (US); Yilian WANG, Los Angeles, CA (US)

(73) Assignee: THE REGENTS OF THE UNIVERSITY OF CALIFORNIA, Oakland, CA (US)

(21) Appl. No.: 18/573,392

(22) PCT Filed: Jun. 23, 2022
(86) PCT No.: PCT/US2022/034797

§ 371 Date: Dec. 21, 2023

Related U.S. Application Data
(60) Provisional application No. 63/214,213, filed on Jun. 23, 2021.
(60) Provisional application No. 63/316,313, filed on Mar. 3, 2022.

(51) Int. Cl. G01N 33/543; B01L 3/00; C12M 1/00; C12M 3/06; G01N 33/569

(52) U.S. Cl. CPC G01N 33/54333; B01L 3/502761; C12M 23/16; C12M 47/04; G01N 33/569; B01L 2200/0652; B01L 2200/16; B01L 2400/043

(57) ABSTRACT

The present embodiments relate generally to a method and apparatus of capturing and releasing magnetically activated cells. Embodiments include a microfluidic device capable of capturing a large array of magnetically functionalized cells and releasing any one specific captured cell without releasing the rest of the cells. Methods and devices according to embodiments provide an improvement over current microfluidic cell sorting approaches due to the use of a simple mechanism, which simultaneously allows for high selectivity, and high throughput potential.

### CROSS-REFERENCE TO RELATED APPLICATIONS

The present application claims priority to U.S. Provisional Patent Application No. 63/214,213 filed Jun. 23, 2021 and U.S. Provisional Patent Application No. 63/316,313 filed Mar. 3, 2022, the contents of all such applications being hereby incorporated by reference herein in their entirety.

### STATEMENT OF GOVERNMENT SPONSORED RESEARCH

This invention was made with government support under Grant Number 1160504, awarded by the National Science Foundation. The government has certain rights in the invention.

### TECHNICAL FIELD

The present embodiments relate generally to cell sorting and more particularly to a method and apparatus of capturing and releasing magnetically activated cells.

### BACKGROUND

Cell sorting has become an indispensable technique for a variety of biological and biomedical applications. With the development of fields such as personalized medicine and new breakthroughs in cancer treatments, more modern cell sorting techniques aimed at addressing the problems at hand are needed now more than ever. Magnetically activated cell sorting (MACS) has quickly become one of the most widely used techniques due to its high performance in throughput, selectivity, and ease of use. Recently, there has been much research and development aimed at creating MACS devices in the microfluidic regime. This is due to the many benefits of using such devices. These benefits include low cost, the ability to run fast low volume experiments, and the ability to capture and release cells while simultaneously culturing them within a device.

It is against this technological backdrop that the present Applicant sought to advance the state of the art using technological solutions to problems rooted in this technology.

### SUMMARY

The present embodiments relate generally to a method and apparatus of capturing and releasing magnetically activated cells. Embodiments include a microfluidic device capable of capturing a large array of magnetically functionalized cells and releasing any one specific captured cell without releasing the rest of the cells. Methods and devices according to embodiments provide an improvement over current microfluidic cell sorting approaches due to the use of a simple mechanism, which simultaneously allows for high selectivity, and high throughput potential.

### BRIEF DESCRIPTION OF THE DRAWINGS

These and other aspects and features of the present embodiments will become apparent to those ordinarily skilled in the art upon review of the following description of specific embodiments in conjunction with the accompanying figures, wherein:

FIG. 1 illustrates example aspects of embodiments.

FIG. 2 is a schematic of an example cell sorting device according to embodiments.

FIGS. 3(a), 3(b) and 3(c) illustrate example micromagnet dimensions and two stable magnetization configurations according to embodiments.

FIGS. 4(a) and 4(b) are graphs illustrating aspects of short and long axis magnetization of an example micromagnet according to embodiments.

FIGS. 5(a) and 5(b) illustrate an example procedure for magnetizing the micromagnet according to embodiments.

FIG. 6 illustrates an example configuration of a capture site having a pair of wires intersecting orthogonal to each other underneath each magnet.

FIG. 7 are images of an example microfabricated device according to embodiments.

FIG. 8 illustrates example Capture and Release Test Results according to embodiments.

FIGS. 9(a), 9(b) and 9(c) illustrates aspects of an example method and device according to one possible alternative embodiment.

### DETAILED DESCRIPTION

The present embodiments will now be described in detail with reference to the drawings, which are provided as illustrative examples of the embodiments so as to enable those skilled in the art to practice the embodiments and alternatives apparent to those skilled in the art. Notably, the figures and examples below are not meant to limit the scope of the present embodiments to a single embodiment, but other embodiments are possible by way of interchange of some or all of the described or illustrated elements. Moreover, where certain elements of the present embodiments can be partially or fully implemented using known components, only those portions of such known components that are necessary for an understanding of the present embodiments will be described, and detailed descriptions of other portions of such known components will be omitted so as not to obscure the present embodiments. Embodiments described as being implemented in software should not be limited thereto, but can include embodiments implemented in hardware, or combinations of software and hardware, and vice-versa, as will be apparent to those skilled in the art, unless otherwise specified herein. In the present specification, an embodiment showing a singular component should not be considered limiting; rather, the present disclosure is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present embodiments encompass present and future known equivalents to the known components referred to herein by way of illustration.

As set forth above, the present Applicant recognizes that cell sorting has become an indispensable technique for a variety of biological and biomedical applications. With the development of fields such as personalized medicine and new breakthroughs in cancer treatments, more modern cell sorting techniques aimed at addressing the problems at hand are needed now more than ever.

Among other things, the present Applicant further notes that magnetically activated cell sorting (MACS) has recently become one of the most widely used techniques due to its high performance in throughput, selectivity, and ease of use. Accordingly, there has been much research and development aimed at creating MACS devices in the microfluidic regime. This is due to the many benefits of using such devices. These benefits include low cost, the ability to run fast low volume experiments, and the ability to capture and release cells while simultaneously culturing them within a device.

Based on this recognition among others by the present Applicant, the present embodiments relate generally to a method and apparatus of capturing and releasing magnetically activated cells in a microfluidic environment. Embodiments include a microfluidic device capable of capturing a large array of magnetically functionalized cells and releasing any one specific captured cell without releasing the rest of the cells.

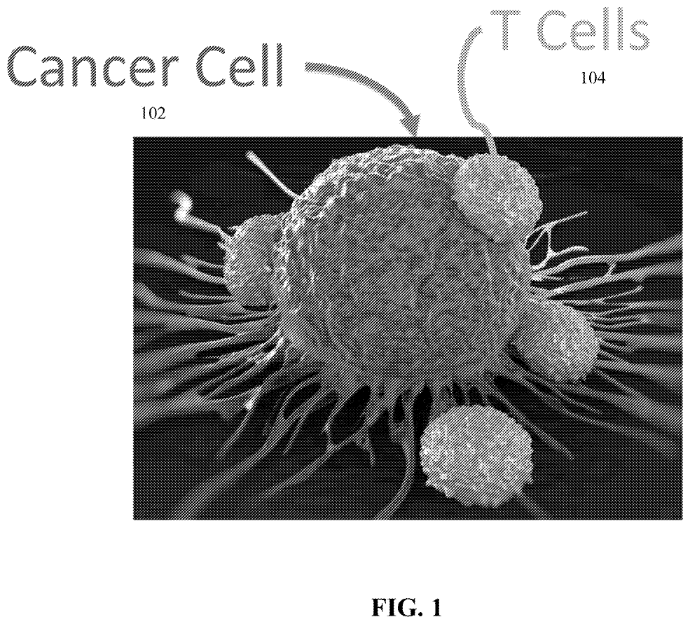

FIG. 1 illustrates example aspects of embodiments. As set forth above, high throughput single cell sorting is essential for the development of new cancer therapies such as CAR T-Cell therapy. One example useful aspect of embodiments is that it provides a device that can magnetically capture and release individual cells such that cancer cells 102 and T-cells 104 can be sorted.

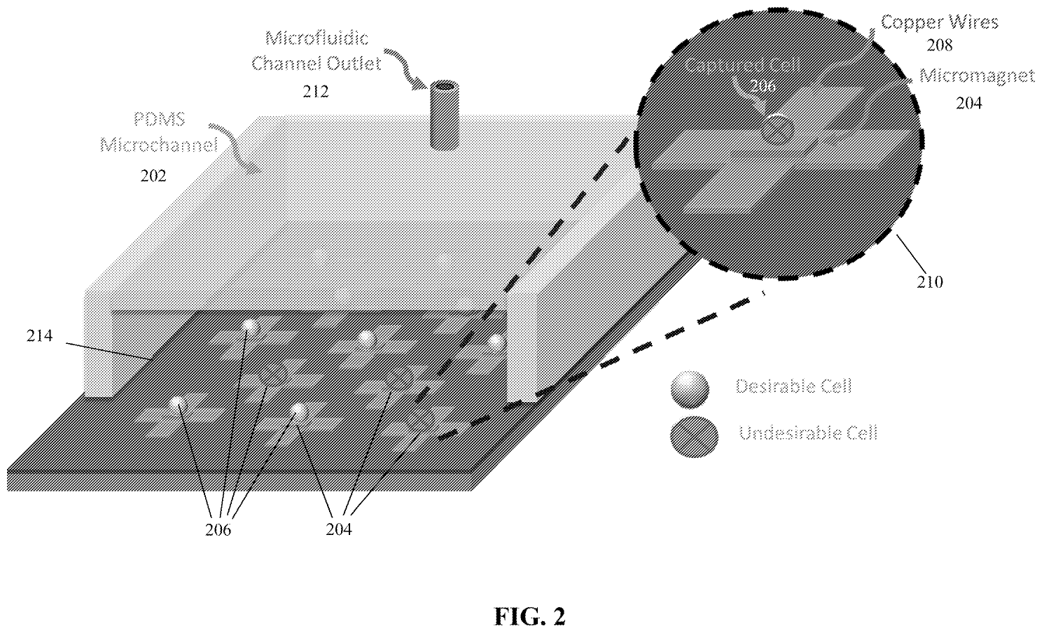

FIG. 2 is a schematic of an example device according to embodiments. As shown, example device 200 includes a microfluidic channel 202 with an array of rectangular micromagnets 204 patterned on the bottom surface of the channel 202 and a microfluidic channel outlet 212 opposite an inlet 214. These micromagnets 204 function as capturing sites for magnetically functionalized cells 206 flowing within the channel. The proposed operation principle of device 200 is now described.

A fluid with a heterogeneous mixture of cells 206 is introduced into the microfluidic channel 202 via inlet 214 where the specific cells to be captured are previously magnetically tagged using conventional antibody-antigen methods. As the mixture of cells flows through the channel, cells labeled with magnetic beads are attracted and captured individually on the patterned micromagnets 204 on the bottom surface of the channel 202.

From this population of captured cells, one can determine if certain cells demonstrate desirable qualities using fluorescent staining methods. Once these desirable cells are identified they can be individually released for collection through outlet 212 without releasing the rest of the captured cells. These collected cells can then be used for further downstream analyses.

According to certain aspects, the device 200 is capable of capturing and releasing individual cells deterministically by essentially turning on and off the magnetic attraction from the capturing sites individually. This is possible due to the rectangular micromagnets having two stable magnetization configurations.

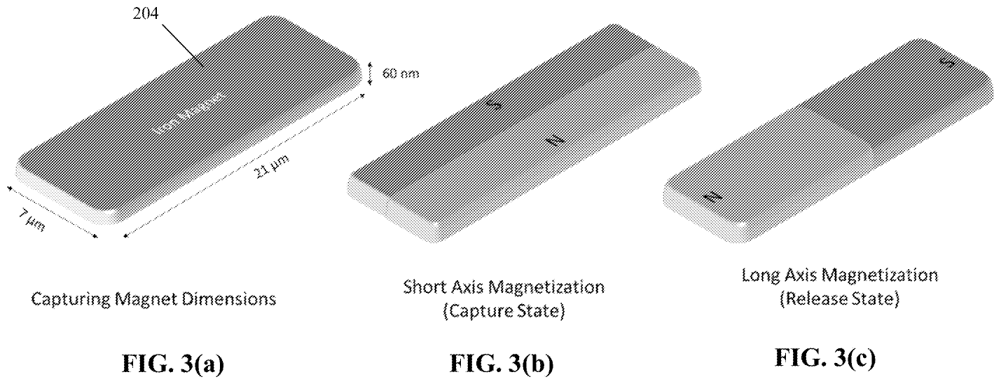

FIG. 3(a) illustrates an example micromagnet that can be used to implement micromagnet 204 as shown in FIG. 2. As shown, in this example, micromagnet 204 has dimensions of about 21 um in a long direction, about 7 um in a short direction, and a height of about 60 nm. In examples, micromagnet is comprised of iron or an iron alloy such as CoFeB. However, these examples are not limiting.

As set forth above, aspects of embodiments include selectively manipulating the magnetization of micromagnets 204 using a short axis and a long axis magnetization direction. When magnetized in the short axis as shown in FIG. 3(b), the attraction force acting on the cells 206 prevents them from flowing along with the fluid thus effectively trapping them. On the other hand, when the rectangular micromagnet is magnetized in the long axis as shown in FIG. 3(c), the magnetic attraction forces acting on the cell are greatly reduced and the drag force from the fluid becomes sufficient to carry the cell downstream for collection. A comparison of the forces generated in the capture and release states is shown below.

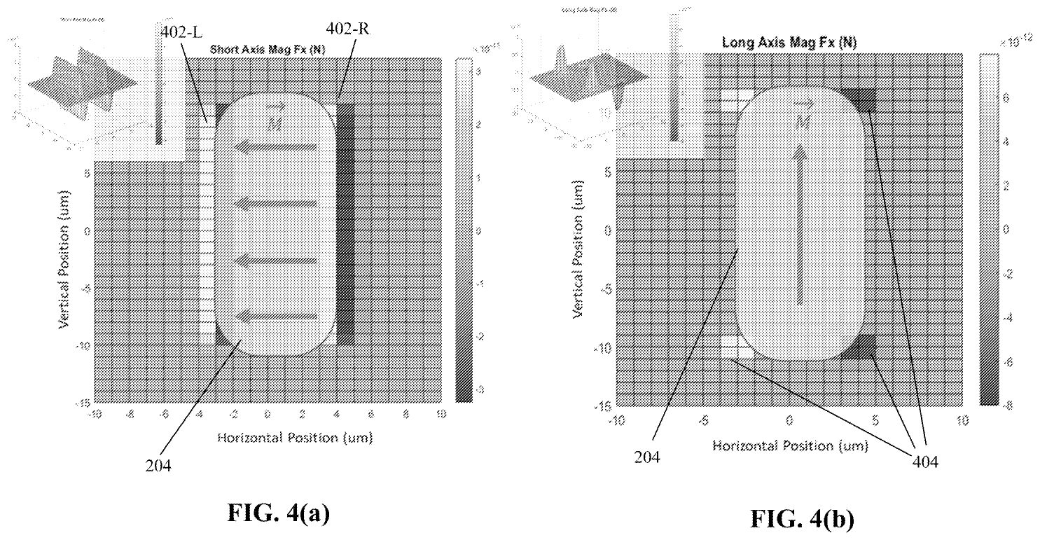

FIGS. 4(a) and 4(b) are graphs illustrating aspects of short and long axis magnetization of an example micromagnet according to embodiments.

As shown in the example of FIG. 4(a), the short axis magnetization exerts around 30 pN of force uniformly along the left and right edges 402-L and 402-R, respectively of the magnet 204 whereas the long axis configuration shown in FIG. 4(b) exerts a maximum of around 6 pN only at the corners 404.

Referring back to FIG. 2, in order to switch between the capture and release state of each capture site 210, a pair of orthogonal wires 208 are configured underneath each rectangular micromagnet 204. By running an electric current in one of these wires, a magnetic field surrounding the wire is generated which will magnetize the rectangular micromagnet 204 in the direction perpendicular to the electric current direction. By using two wires underneath each capture site, it is possible to deterministically magnetize the rectangular micromagnet 204 in either the capture or release state.

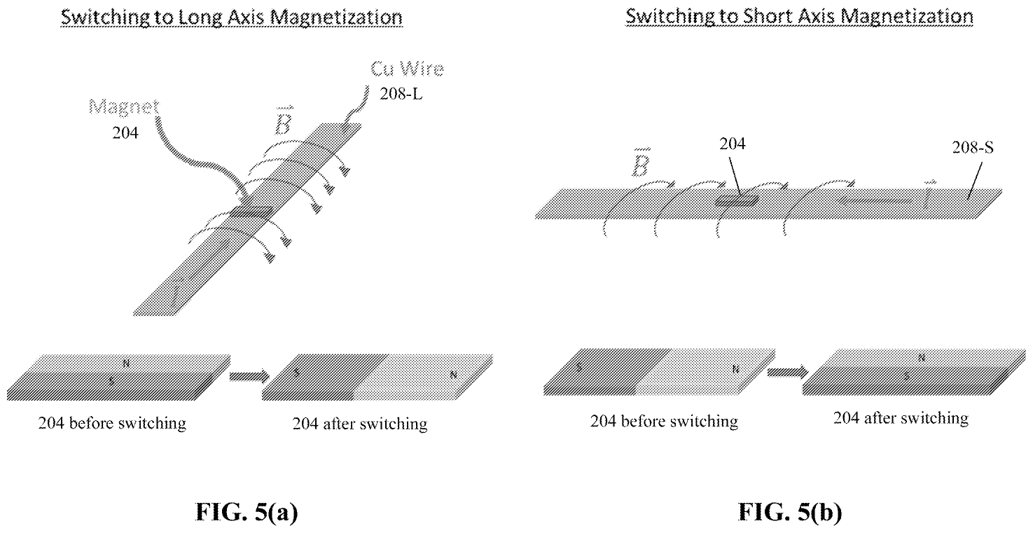

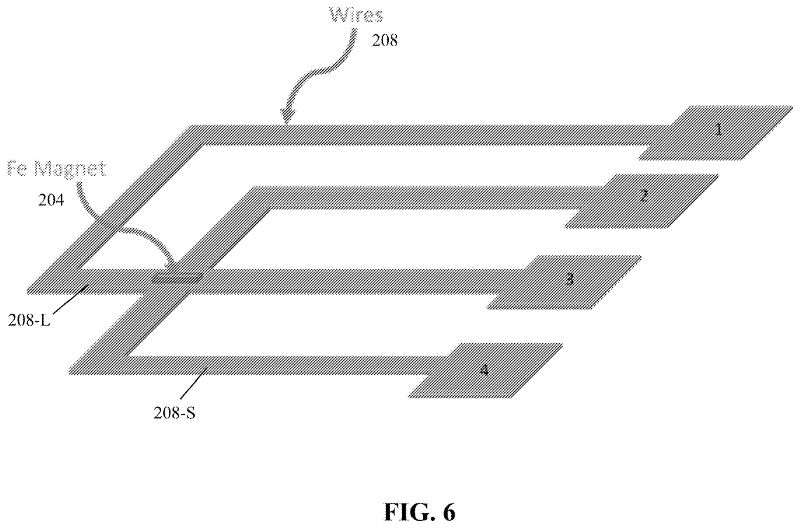

For example, FIG. 5(a) illustrates an example procedure for magnetizing the micromagnet 204 in the long axis (e.g. axis corresponding to a longer length of the rectangular magnet) according to embodiments. As shown, it includes running a current through a copper wire 208-L configured in a long axis direction under magnet 204. This causes magnetic fields B having field lines parallel to a long axis directions around wire 208-L. FIG. 5(b) illustrates an example procedure for short axis magnetization (e.g. axis corresponding to a shorter length of the rectangular magnet) according to embodiments. As shown, it includes running a current through copper wire 208-S configured in a short axis direction under magnet 204. This causes magnetic fields B having field lines parallel to a short axis directions around wire 208-S. As further shown in FIG. 6, to be able to switch between the two states deterministically the two wires 208-S and 208-L are arranged to intersect orthogonal to each other underneath each magnet 204, and further arranged to have terminals 1, 2, 3 and 4 to which electrical power/current can be selectively applied in the desired directions.

In the example device such as device 200 there are two main components that can be subject to careful design optimization. These are the rectangular micromagnets and the wires underneath them.

Accordingly, as shown in FIG. 3(a), in one example, the size of the rectangular micromagnets 204 used to capture the cells in a device such as that shown in FIG. 2 have a size of 7 um×21 um×60 nm. These dimensions allow capturing and releasing a single cell per micromagnet. If they were any smaller, they may not generate enough capture force and if they were any larger, there would be a risk of capturing more than a single cell per capturing site. It is preferable for the micromagnets to comprise iron since it is an easily available material, and it has a high saturation magnetization which is directly proportional to the amount of magnetic attraction it can exert on magnetically functionalized cells.

In one example, the wires 208 are made of copper and their dimensions are 30 um in width and 1 um thick. The width of the wires was made large enough to allow for easy patterning of the micromagnets on the surface of the wire and also so that the magnetic field generated from the current in the wire would magnetize the rectangular micromagnet uniformly. This width also had to be simultaneously minimized to reduce the total footprint of each capture site. The thickness of wire was set to 1 um because in some embodiments, this is the maximum thickness of copper that can be reliably deposited using e-beam evaporation. It is preferable to obtain the thickest wire possible because this maximizes the amount of current that could be run through the wire which would allow generating the necessary fields needed to magnetize the micromagnets into the capture or release state. This thickness of wire also minimizes the resistance which allows minimizes the voltages required to operate the device and also minimizes the heat produced by the wires during operation.

The voltages and currents applied to the wires 208 via associated contacts (e.g. terminals 1-4 as shown in FIG. 6) depend on the geometry and dimensions of magnets 204 and the particular cell sorting or other application, as will be appreciated by those skilled in the art. In some embodiments such as the device 200 shown above, a pulse current of around 3 amps has been found to be sufficient to magnetize the rectangular iron structures in either the capture (short axis) and release (long axis) magnetization states. This and other example current magnitudes can be calculated by modeling the magnetic fields produced around the wire under different currents and also by modeling and measuring the required magnetic field magnitude necessary to fully magnetize the capture structures in either configuration. Moreover, for an example device 200 such as that described herein, the voltage required to induce a 3 Amp current is around 40V but this is subject to change since it depends on the geometry of the wires which can be changed to accommodate more capturing sites.

It should be noted that one possible method of releasing the captured cells described herein is by demagnetizing the structures rather than magnetizing them in the release state. If this approach is used, in order to magnetize the structures, around 3 Amp pulse current is used. However, in order to demagnetize a current pulse of only around 1.5 amp flowing in the opposite direction to the original magnetizing current can be used.

It should be further apparent that the voltages and currents can be generated by any suitable power source, which can be further controlled to selectively apply them to any desired capture sites so as to individually capture and release cells at such sites by any suitable controller circuitry, switches, etc. Such circuitry etc. can be further controlled by any combination of automatic and manual means. The details of such power sources and control circuitry and methods are omitted here for sake of clarity of the present embodiments.

One example process for fabricating a device such as device 200 is now herein described. The process includes cleaning a high resistivity Si wafer with acetone methanol and isopropanol. The cleaned wafer is subject to a dehydration bake at 150 C for 15 minutes. This is followed by a process to spin coat HMDS on the wafer, for example at 3000 rpm for 60s. Next is a process to spin coat an AZnLOF2020 photoresist on the wafer, for example at 3000 rpm for 60 s. Then a soft bake at 110 C is performed for 1 minute.

The resist is then exposed for 5.6 s using 12 mW lamp in Karl Suss Aligner using a pattern for the wires 208 and associated contacts. A post exposure bake at 110 C is then performed for 1 minute. The photoresist is then developed using AZ300MIF Developer for 90 seconds. Descum is then performed for 1 minute using Oxygen Plasma Asher at 80 W and 50 C. Wires 208 are then formed by depositing Ti(5 nm)/Cu(1 μm)/Ti(5 nm) using a CHA e-beam evaporator. In this example, titanium is used here as an adhesion layer and a capping layer to prevent oxidation. Liftoff photoresist using NMP is then performed overnight. An ultrasonic bath can be used to speed up liftoff.

The photolithography steps described above for wires 208 can then be repeated to pattern rectangular capture sites. Thereafter, the magnets 204 can be formed by depositing Ti(5 nm)/Fe(60 nm)/Ti(5 nm). The thickness of the Fe can be modified to increase the capture strength of the magnets. Liftoff photoresist using NMP can then be performed overnight.

A process is then performed to deposit a passivating SiO₂ layer using STS PECVD system. This layer can have a thickness of about 2 μm. This can be verified using the Nanospec spectroscopic reflectometry system. The photolithography steps described above can be repeated to pattern the SiO₂ etching mask. Unwanted SiO₂ can then be removed from the Copper contacts using BOE for 30 minutes. Finally the resist can be removed using NMP and an ultrasonic bath.

Device 200 fabrication can be completed by bonding a polydimethylsiloxane (PDMS) microfluidic channel having at least one inlet and one outlet onto the substrate using oxygen plasma bonding. The channel can have dimensions depending on the number of cell sorting sites that are desired to be included within the channel.

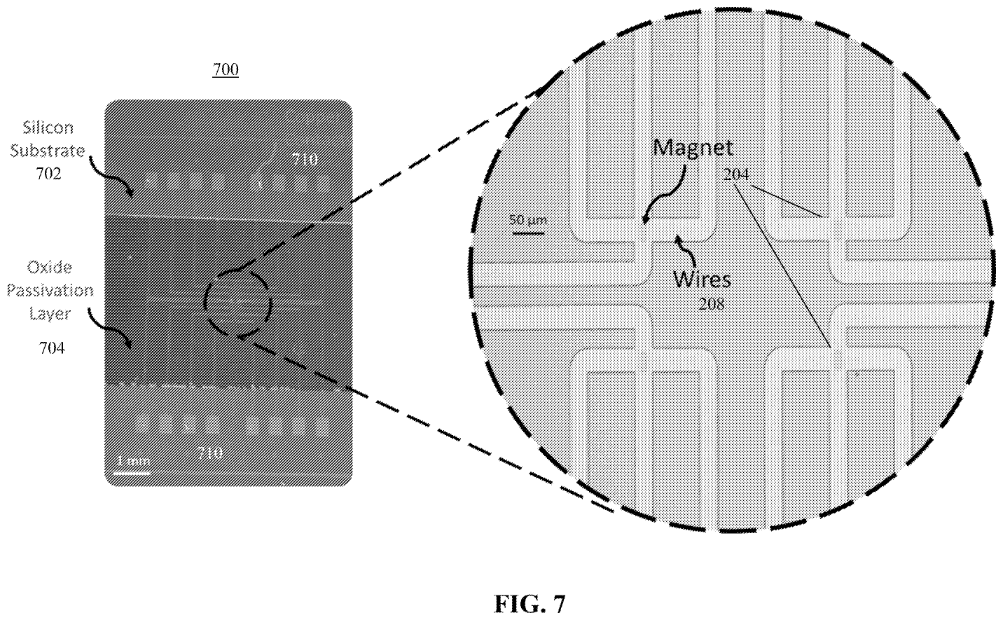

Images of an example microfabricated device 700 using a process such as that described above are shown in FIG. 7, including silicon substrate 702 and oxide passivation layer 704. This example device 700 includes four magnets 706 having underlying wires 708 and exposed copper wire contacts 710.

After fabricating a proof-of-concept device such as that shown in FIG. 7, the present Applicants used this device to experimentally verify whether it could capture and release magnetic beads using the mechanism described above. The experiment was conducted using a 2×2 array of capture sites. All four micromagnets were first initialized in the capture state by running a current in the vertical direction and allowing magnetic beads to flow into the channel. Once beads had been captured on all four capture sites, a current was run in the horizontal direction under micromagnets 1 and 4 to switch them into the release state. It was observed that almost all the beads were completely released upon doing this.

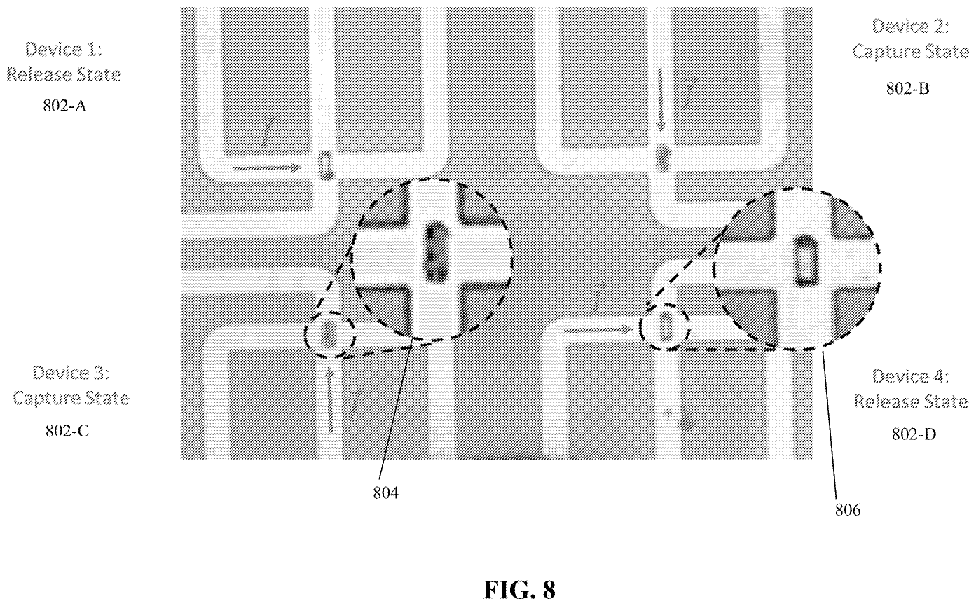

Example results of this test are shown in FIG. 8. As described above, this includes configuring magnet 1 into a release state 802-A, magnet 2 into a capture state 802-B, magnet 3 into a capture state 802-C, and magnet 4 into a release state 802-D. As further shown in the blowup 804 of capture state 802-C, the captured beads are attached to magnet 3, while in the blowup 806 of capture state 802-D, the captured beads are released from magnet 3.

A device described above has been fabricated and experimentally verified to achieve both capture and release of single cells. However, many alternatives of this device and operation procedure can be made to massively array these capture sites while retaining the single cell release capabilities. For example, alternatives can turn on and off the magnetic attraction from the capturing sites using a slightly different magnetization method.

One example includes using a single wire switching mechanism. In a first step this embodiment magnetizes the micromagnet to saturation in the short axis using a strong current. Then in in order to demagnetize the micromagnet this embodiment runs a smaller current in the opposite direction to generate a field on the order of the coercivity He of the magnetic structure.

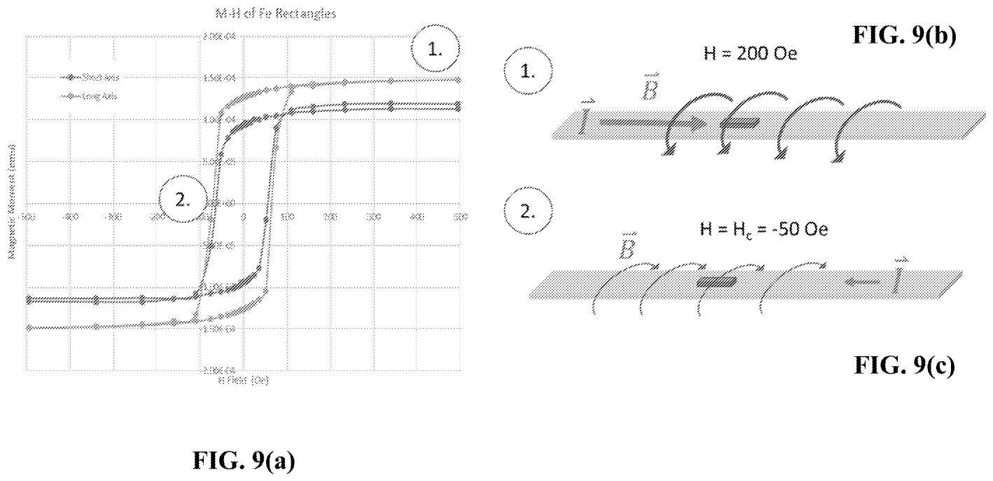

Accordingly, instead of using two wires to magnetize the rectangular capturing sites into the short axis and the long axis as described above, this alternative can magnetize and demagnetize the structures using a single wire. In order to magnetize the capturing site, as shown in FIG. 9(b), this embodiment can run a large current that produces a sufficiently high magnetic field that is able to magnetize the structure in the capture state indefinitely. As shown in FIG. 9(c), the structure can thereafter be demagnetized by running a smaller current in the opposite direction. This will produce a field directly opposing the initial magnetization state which will act to demagnetize the sample. The strength of the current is reduced because a current of the same magnitude in the opposite direction this would result in the structure being fully magnetized again in the opposite direction. We have found that in order to successfully demagnetize our capturing sites we must run a current in the opposite direction and with about half the strength of the initial current used to magnetize the capturing site. This is further illustrated in the graph in FIG. 9(a).

According to certain additional aspects, embodiments can include other techniques to change the magnetization of the magnet from the capture state to the release state in an efficient manner. In some alternative embodiments a state-of-the-art magnetic manipulation method known as Spin Orbit Torque (SOT) was used. In this approach, a heavy metal (Tantalum) wire is deposited in contact with the capturing micromagnet and whenever an electric current flows in this heavy metal wire a spin current is injected into the magnet. This spin current will change the magnetic state of the magnet to a configuration perpendicular to the direction of the electric current in the heavy metal. We chose this approach because it has proven to be a very fast and efficient method of magnetization switching in research areas related to magnetic memory. SOT also allows us to easily change the magnetic state of individual magnets, whereas this can be significantly more difficult using external magnets or using strain mediated methods. In general, SOT works by running an electric current through a heavy metal conductor (such as tantalum, platinum or tungsten) that is in contact with the magnetic microstructure. This electric current becomes spin polarized within the heavy metal, which in turn injects a magnetic spin a current into the microstructure which will then change the magnetic state. Given that a particular electric current direction induces a specific magnetization direction we have two perpendicular wires placed underneath the magnetic microstructure to be able to switch between the capture and release state deterministically.

While this magnetic switching method is very energy efficient, it suffers from several limitations. The most important limitation being that it can only switch the magnetic state of very thin microstructures since the SOT effect decays with the thickness of the magnet. This becomes a problem because, for cell sorting applications, we need magnets that are able tocan generate at the capture force at a sufficient distance to attract all the magnetically functionalized cells flowing within the microchannel. The force generated by a magnet is directly correlated to its size, the bigger the magnet the bigger the attraction force it will generate. For this reason, it has been envisaged to change the magnetization switching method from SOT to a more classical switching method using the magnetic fields inherently generated from an electric current. The fields produced by an electric current are known as Oersted fields and given a high enough current they are able to switch the magnetic state of thicker magnetic microstructures.

In one additional alternative embodiment of the device, Oersted fields can be used to switch the magnets instead of using SOT. The idea behind Oersted switching is a lot simpler than that of SOT. This alternative includes just running a high enough current in the wires underneath the magnetic microstructures to produce a strong field surrounding the wires which can, in turn, magnetize the microstructures into either the capture or release state. As far as the design of the device goes, the geometry is basically the same as the SOT approach with a few exceptions. The wires used are no longer made of heavy metals but rather made of copper given its low resistivity. This alternative also greatly increased the thickness of the wires in order to lower the resistance allowing us to run high enough currents to produce strong magnetic fields that can switch the magnetic state of the microstructures. This new approach allows the device to easily change the magnetic state of thicker magnetic microstructures which allows it to capture and release a larger number of single cells more efficiently.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably coupleable,” to each other to achieve the desired functionality. Specific examples of operably coupleable include but are not limited to physically mateable and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

Although the present embodiments have been particularly described with reference to preferred examples thereof, it should be readily apparent to those of ordinary skill in the art that changes and modifications in the form and details may be made without departing from the spirit and scope of the present disclosure. It is intended that the appended claims encompass such changes and modifications.

What is claimed is:

1. A method comprising:
capturing and releasing magnetically activated cells using micromagnets placed within a microfluidic device, and controlling magnetization states of the micromagnets using one or more wires underneath the micromagnets.

2. The method of claim 1 wherein a high enough current is run in the wires underneath the micromagnets to produce a strong field surrounding the wires so as to magnetize the micromagnets into either a capture or release state.

3. The method of claim 1, further comprising:
configuring a pair of wires that intersect underneath each of the micromagnets.

4. The method of claim 3, wherein the micromagnets are rectangular in shape, and controlling the magnetization includes selectively applying a current in only one of the pair of wires.

5. The method of claim 4, wherein when applying the current in a first one of the pair of wires, the micromagnets are configured to capture the magnetically activated cells.

6. The method of claim 5, wherein when applying the current in a second one of the pair of wires, the micromagnets are configured to release the magnetically activated cells.

7. An apparatus configured to capture and release magnetically activated cells, comprising:
a microfluidic device;
micromagnets placed within the microfluidic device; and
wires configured underneath the micromagnets.

8. The apparatus of claim 7, wherein the wires are comprised of copper.

9. The apparatus of claim 7, wherein a thickness of the wires is configured to lower the resistance to run high enough currents to produce strong magnetic fields that can switch the magnetic state of the micromagnets.

10. The apparatus of claim 7, wherein the wires include a pair of wires that intersect underneath each of the micromagnets.

11. The apparatus of claim 10, wherein the micromagnets are rectangular in shape, and the magnetization of the micromagnets is controlled by selectively applying a current in only one of the pair of wires.

12. The apparatus of claim 11, wherein when applying the current in a first one of the pair of wires, the micromagnets are configured to capture the magnetically activated cells.

13. The apparatus of claim 12, wherein when applying the current in a second one of the pair of wires, the micromagnets are configured to release the magnetically activated cells.

14. The apparatus of claim 7, wherein the microfluidic device includes a channel having an inlet and an outlet for receiving and releasing the magnetically activated cells, respectively.

15. The apparatus of claim 14, wherein the micromagnets are configured on a bottom surface of the channel.
